(19) United States
(12) Patent Application Publication
(10) Pub. No.: US 2002/0193422 A1
(43) Pub. Date: Dec. 19, 2002

(54) Anthranilamides and methods of their use

(76) Inventors: Joachim Brendel, Bad Vilbel (DE); Bernard Pirard, Frankfurt (DE)

Correspondence Address: Finnegan, Henderson, Farabow,, Garrett & Dunner, L.L.P., 1300 I Street, N.W., Washington, DC, 20005-3315, US

(21) Appl. No.: 10/132,184

(22) Filed: Apr. 26, 2002

(30) Foreign Application Priority Data
Apr. 28, 2001 (DE) 10121002.7

(51) Int. Cl. A61K 31/381; A61K 31/277; A61K 31/343; A61K 31/24; A61K 31/18; A61K 31/195

(52) U.S. Cl. 514/447; 514/471; 514/605; 514/562; 514/534; 514/522

(57) ABSTRACT

The presently disclosed invention relates to compounds of general formula I:

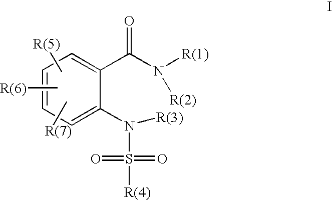

and/or any pharmaceutically acceptable salts thereof. The present invention discloses methods of preparing such compounds, and their use as medicaments having a K⁺-channel-blocking activity to treat and/or prevent K⁺-channel-mediated diseases, such as cardiac arrhythmias, reentry arrhythmias, supraventricular arrhythmias, atrial fibrillation or atrial flutter.

The invention generally relates to compounds of formula I,

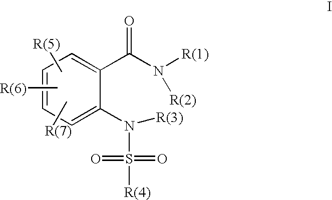

in which R(1), R(2), R(3), R(4), R(5), R(6) and R(7) have the meanings stated below, and methods of their preparation and use as medicaments. The compounds generally exhibit a K⁺-channel-blocking action. Consequently, the compounds are useful for treating and/or preventing K⁺-channel-mediated diseases, such as arrhythmia.

Some of the compounds of formula I described herein have been reported in the scientific literature. For example, compounds A and B, infra, were described in FEBS Letters (1981) 421:217-220, as serine protease inhibitors. Compounds C and D, infra, and similar derivatives, were described in J. Med. Chem. (1968) 11:777-787, as precursors for the synthesis of tetrahydroisoquino[2,1-d][1,4]benzodiazepines. These publications, however, provide no teaching or suggestion that the presently disclosed compounds have a K⁺-channel-blocking action and may be useful as medicaments for the therapy and prophylaxis of K⁺-channel-mediated diseases, such as arrhythmia.

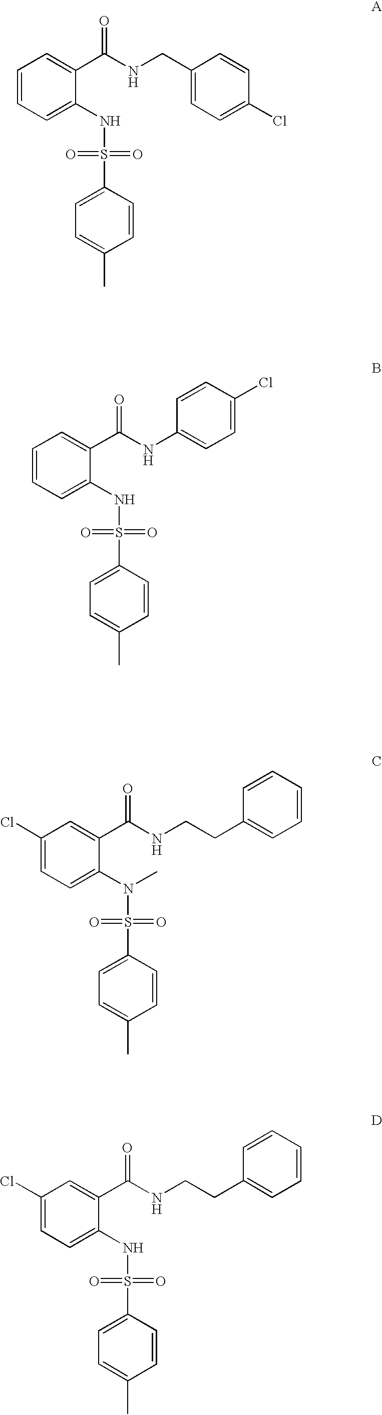

EP-A-686 625 describes anthranilic acid derivatives and their use as cGMP phosphodiesterase inhibitors. Most of the 144 compounds described in this reference contain carbonyl groups, which differ from the sulfonyl groups in the presently disclosed compounds. Three of the examples provide a sulfonylamino substituent (see, e.g., Example 131 in EP 686 625), which is structurally similar to compound E, infra. However, the reference provides no teaching or suggestion that such compounds function as phosphodiesterase inhibitors or that they may be used as antiarrhythmic agents.

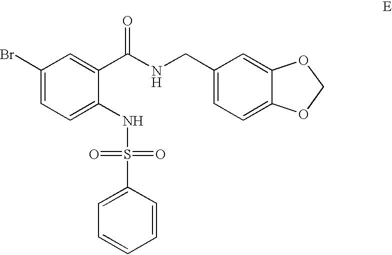

EP-A-947 500 claims a large, heterogeneous group of compounds which reportedly act as prostaglandin E2 antagonists or agonists. Most of the described anthranilic acid derivatives contain a free carboxylic acid function, thus differing from the presently disclosed compounds. Only compound F (see example 1 in EP 947 500) is similar to the presently disclosed compounds. However, it is not clear from the reference whether this compound actually has the prostaglandin E2 activity described for the other compounds. Moreover, the reference does not teach or suggest the use of such compounds as antiarrhythmic agents.

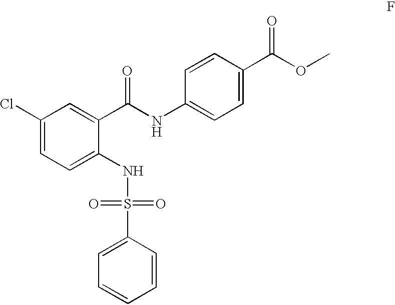

DE-A-32 25 966 describes alkylsulfonyl-substituted anthranilic acids, such as compound G, and their use as fluorescent dyes.

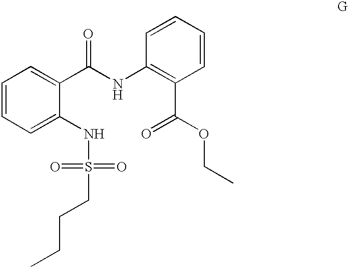

The international application WO 00 78 145 describes anthranilanilides, such as the bromine derivative H, and their use for the treatment of arteriosclerosis by antagonism of the MSR receptor.

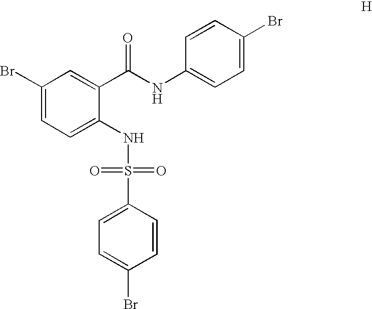

In view of these publications, the present inventors have now surprisingly found that the anthranilamides of formula I, some of which are known, are potent inhibitors of the Kv1.5 potassium channel and thus block a potassium current referred to as an “ultra-rapidly activating delayed rectifier” in the human atrium. The compounds are therefore useful as novel antiarrhythmic active substances, for example, in the treatment and prophylaxis of atrial arrhythmias such as atrial fibrillation (AF) or atrial flutter.

Atrial fibrillation (AF) and atrial flutter are the most frequent and persistent cardiac arrhythmias. Their occurrence increases with increasing age and frequently leads to other fatal symptoms, such as stroke. AF affects about 1 million Americans annually and leads to more than 80,000 strokes every year in the USA. The customary antiarrhythmic agents of classes I and III typically reduce the rate of AF occurrence, but owing to their potential proarrhythmic side effects, can only be used in a limited manner. Consequently, there is a considerable medical necessity for the development of better medicaments for the treatment of atrial arrhythmias (S. Nattel (1995) “Newer developments in the management of atrial fibrillation,” Am. Heart J. 130:1094-1106).

It was shown that most supraventricular arrhythmias are subject to “reentry” excitatory waves. Such reentries occur when the heart tissue exhibits slow conductivity and, at the same time, very short refractory periods. Increasing the myocardial refractory period by lengthening the action potential is a recognized mechanism for terminating arrhythmias or preventing their occurrence (T. J. Colatsky et al (1990), “Potassium channels as targets for antiarrhythmic drug action,” Drug Dev. Res. 19:129-140). The length of the action potential is substantially determined by the extent of repolarizing K⁺ flows which flow out of the cell via various K⁺ channels. The “delayed rectifier” I_{K}, which consists of 3 different components: IKᵣ, IKₛ and IKᵤᵣ, is ascribed particular importance in this process.

Most known class III arrhythmic agents (e.g., dofetilide, E4031 and d-sotalol) predominantly or exclusively block the rapidly-activating potassium channel IKᵣ, which is detectable both in cells of the human ventricle and in the atrium. However, it has been found that at low or normal heart rates these compounds increase the risk of arrhythmias, in particular, arrhythmias which are referred to as “torsades de pointes” (D. M. Roden (1993) “Current status of class III antiarrhythmic drug therapy,” Am. J. Cardiol. 72:44B-49B). In addition to this increased risk, which can be fatal, the efficacy of I_{Kr} blockers declines at the lower heart rates experienced during conditions of tachycardia, precisely when the effective action of these blockers is needed most.

While some of these disadvantages can possibly be overcome by blockers of the slowly activating component (IKₛ), their efficacy has not been proven to date since no clinical studies with IKₛ channel blockers are known.

The “ultra-rapidly” activating and very slowly deactivating component of the delayed rectifier is termed IKᵤᵣ (=ultra-rapidly activating delayed rectifier). This corresponds to the Kv1.5 channel, and plays a major role in the repolarization time in the human atrium. Compared with the inhibition of IKᵣ or IKₛ, inhibition of the IKᵤᵣ potassium outward current is a more effective method for lengthening the atrial action potential, thus terminating or preventing atrial arrhythmias. Mathematical models of human action potential suggest that the positive effects of blocking the IKᵤᵣ should be particularly pronounced under the pathological conditions of chronic atrial fibrillation (M. Courtemanche, R. J. Ramirez, S. Nattel (1999) “Ionic targets for drug therapy and atrial fibrillation-induced electrical remodeling: insights from a mathematical model,” Cardiovascular Research, 42:477-489).

In contrast to IKᵣ and IKₛ, which occur in the human ventricle, IKᵤᵣ plays an important role in the human atrium but not the ventricle. Thus, unlike IKᵣ and IKₛ blockers, the risk of a proarrhythmic effect from IKᵤᵣ blockers in the ventricle is not a concern (Z. Wang et al (1993) “Sustained Depolarization-Induced Outward Current in Human Atrial Myocytes,” Circ. Res., 73:1061-1076; G. R. Li et al. (1996) “Evidence for Two Components of Delayed Rectifier K⁺-Current in Human Ventricular Myocytes,” Circ. Res., 78:689-696; G. J. Amos et al. (1996) “Differences between outward currents of human atrial and subepicardial ventricular myocytes,” J. Physiol., 491:31-50).

However, antiarrhythmic agents which act via selective blocking of the IKᵤᵣ current or Kv1.5-channel have not been commercially available to date. Although a blocking effect on the Kv1.5 channel has been described for numerous pharmaceutical active substances (e.g., tedisamil, bupivacaine or sertindole), the Kv1.5 block here is only a side effect to the intended main effects of the substances.

WO 98 04 521 and WO 99 37 607 describe aminoindanes and aminotetrahydrona-phthalenes as potassium channel blockers which block the Kv1.5 channel. Structurally related aminochromanes are likewise described as Kv1.5 blockers in WO 00 12 077. In WO 99 62 891, thiazolidinones which block the potassium channel are also described. The applications WO 98 18 475 and WO 98 18 476 describe the use of various pyridazinones and phosphine oxides as antiarrhythmic agents which are said to act by blocking the IKᵤᵣ. However, the same compounds were known to be immuno-suppressives (WO 96 25 936). All compounds described in these publications are completely different structurally from the presently disclosed compounds of the invention. Furthermore, the present inventors are not aware of any clinical data for the compounds disclosed in these publications. Since experience has shown that only a small proportion of active substances from preclinical research successfully overcome all clinical hurdles to gain approval as medicaments, there is still a need in the art for promising new compounds.

Owing to their blocking properties with respect to the Kv1.5 channel, the anthranilamides of the present invention are useful as novel antiarrhythmic agents that have a particularly advantageous safety profile. In particular, the compounds are suitable for the treatment of supraventricular arrhythmias, e.g., atrial fibrillation or atrial flutter.

The presently disclosed compounds can be used for terminating existing atrial fibrillation or atrial flutter in order to restore the sinus rhythm (cardioversion). In addition, the substances reduce the susceptibility to developing further fibrillation events (e.g., retention of the sinus rhythm, prophylaxis).

The present invention relates to the use of compounds of formula I

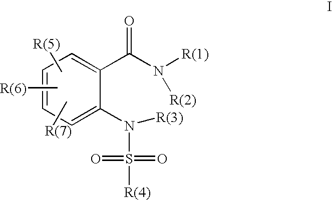

in which:

R(1) is (CH₂)ₓ—R(8)

x is 0, 1, 2, 3, 4 or 5;

R(8) is phenyl, thienyl or furanyl, phenyl, thienyl and furanyl being unsubstituted or substituted by 1, 2 or 3 substituents chosen from among F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;

R(2) is hydrogen or alkyl having 1, 2 or 3 carbon atoms;

R(3) is hydrogen or alkyl having 1, 2 or 3 carbon atoms;

R(4) is alkyl having 3, 4, 5, 6 or 7 carbon atoms, cycloalkyl having 3, 4, 5, 6 or 7 carbon atoms, phenyl, naphthyl or heteroaryl, phenyl, naphthyl and heteroaryl being unsubstituted or substituted by 1, 2 or 3 substituents chosen from among F, Cl, Br, I, CF₃, OCF₃, NO₂, COOMe, CONH₂, COMe, NH₂, OH, alkyl having 1, 2, 3 or 4 carbon atoms, alkoxy having 1, 2, 3 or 4 carbon atoms, dimethylamino, methylsulfonyl and methylsulfonylamino;

R(5), R(6) and R(7), independently of one another, are chosen from among F, Cl, Br, I, CF₃, OCF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;

and their pharmaceutically acceptable salts. The compounds of formula I, and their pharmaceutically acceptable salts, are useful as medicaments having a K⁺-channel-blocking action for treating and preventing K⁺-channel-mediated diseases.

In one embodiment, the compounds of formula I include those in which:

R(1) is (CH₂)ₓ—R(8)

x is 1, 2, 3 or 4;

R(8) is phenyl, thienyl or furanyl, phenyl, thienyl and furanyl being unsubstituted or substituted by 1, 2 or 3 substituents chosen from among F, Cl, Br, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;

R(2) is hydrogen or alkyl having 1, 2 or 3 carbon atoms;

R(3) is hydrogen or alkyl having 1, 2 or 3 carbon atoms;

R(4) is alkyl having 4, 5, 6 or 7 carbon atoms, cycloalkyl having 5, 6 or 7 carbon atoms, phenyl or heteroaryl, phenyl and heteroaryl being unsubstituted or substituted by 1, 2 or 3 substituents chosen from among F, Cl, Br, CF₃, OCF₃, NO₂, COOMe, CONH₂, COMe, NH₂, OH, alkyl having 1, 2, 3 or 4 carbon atoms, alkoxy having 1, 2, 3 or 4 carbon atoms, dimethylamino, methylsulfonyl and methylsulfonylamino;

R(5), R(6) and R(7), independently of one another, are chosen from among F, Cl, Br, CF₃, OCF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;

and their pharmaceutically acceptable salts.

In another embodiment, the compounds of formula I include those in which:

R(1) is (CH₂)ₓ—R(8)

x is 1, 2, 3 or 4;

R(8) is phenyl, thienyl or furanyl, phenyl, thienyl and furanyl being unsubstituted or substituted by 1, 2 or 3 substituents chosen from among F, Cl, Br, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;

R(2) is hydrogen or alkyl having 1, 2 or 3 carbon atoms;

R(3) is hydrogen or alkyl having 1 or 2 carbon atoms;

R(4) is phenyl, phenyl being unsubstituted or substituted by 1, 2 or 3 substituents chosen from among F, Cl, Br, CF₃, OCF₃, NO₂, COOMe, CONH₂, COMe, NH₂, OH, alkyl having 1, 2, 3 or 4 carbon atoms, alkoxy having 1, 2, 3 or 4 carbon atoms, dimethylamino, methylsulfonyl and methylsulfonylamino;

R(5), R(6) and R(7), independently of one another, are chosen from among F, Cl, Br, CF₃, OCF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino; and their pharmaceutically acceptable salts.

In yet another embodiment, the compounds of formula I include those in which:

R(1) is (CH₂)ₓ—R(8)

x is 1 or 2;

R(8) is phenyl, thienyl or furanyl, phenyl, thienyl and furanyl being unsubstituted or substituted by 1, 2 or 3 substituents chosen from among F, Cl, Br, CF₃, OCF₃, CN, COOMe, CONH₂, COMe, alkyl having 1, 2 or 3 carbon atoms, alkoxy having 1, 2 or 3 carbon atoms, sulfamoyl, methylsulfonyl and methylsulfonylamino;

R(2) is hydrogen or alkyl having 1 or 2 carbon atoms;

R(3) is hydrogen;

R(4) is phenyl, phenyl being unsubstituted or substituted by 1, 2 or 3 substituents chosen from among F, Cl, Br, CF₃, OCF₃, COMe, alkyl having 1, 2 or 3 carbon atoms and alkoxy having 1, 2 or 3 carbon atoms;

R(5), R(6) and R(7), independently of one another, are chosen from among F, Cl, Br, CF₃, OCF₃, CN, COOMe, CONH₂, COMe, alkyl having 1, 2 or 3 carbon atoms, alkoxy having 1, 2, 3 or 4 carbon atoms, dimethylamino, sulfamoyl, methylsulfonyl and methylsulfonylamino;

and their pharmaceutically acceptable salts.

The invention relates to the use of the compounds of formula I and/or pharmaceutically acceptable salts thereof, as medicaments for (i) the therapy or prophylaxis of cardiac arrhythmias which can be eliminated by lengthening the action potential; (ii) the therapy or prophylaxis of reentry arrhythmias; (iii) the therapy or prophylaxis of supraventricular arrhythmias; (iv) the therapy or prophylaxis of atrial fibrillation or atrial flutter; or (v) the termination of atrial fibrillation or atrial flutter (cardioversion).

In the compounds of formula I, alkyl radicals and alkoxy radicals may be straight-chain or branched. Examples of alkyl radicals include methyl, ethyl, n-propyl, isopropyl, n-butyl, isobutyl, sec-butyl, tert-butyl, n-pentyl, isopentyl, neopentyl, n-hexyl, 3,3-dimethylbutyl and heptyl.

Cycloalkyl radicals may likewise be branched. Examples of cycloalkyl radicals having 3 to 7 carbon atoms include cyclopropyl, cyclobutyl, 1-methylcyclopropyl, 2-methylcyclopropyl, cyclopentyl, 2-methylcyclobutyl, 3-methylcyclobutyl, cyclopentyl, cyclohexyl, 2-methylcyclohexyl, 3-methylcyclohexyl, 4-methylcyclohexyl, cycloheptyl etc.

Heteroaryl radicals include 2- or 3-thienyl, 2- or 3-furanyl, 1-, 2- or 3-pyrrolyl, 1, 2-, 4- or 5-imidazolyl, 1-, 3-, 4- or 5-pyrazolyl, 1,2,3-triazol-1-, -4- or -5-yl, 1,2,4-triazol-1-, -3- or -5-yl, 1- or 5-tetrazolyl, 2-, 4- or 5-oxazolyl, 3-, 4- or 5-isoxazolyl, 1,2,3-oxadiazol-4- or -5-yl, 1,2,4-oxadiazol-3- or -5-yl, 1,3,4-oxadiazol-2-yl or -5-yl, 2-, 4- or 5-thiazolyl, 3-, 4- or 5-isothiazolyl, 1,3,4-thiadiazol-2- or -5-yl, 1,2,4-thiadiazol-3- or -5-yl, 1,2,3-thiadiazol-4- or -5-yl, 2-, 3- or 4-pyridyl, 2-, 4-, 5- or 6-pyrimidinyl, 3- or 4-pyridazinyl, pyrazinyl, 1-, 2-, 3-, 4-, 5-, 6- or 7-indolyl, 1-, 2-, 4- or 5-benzimidazolyl, 1-, 3-, 4-, 5-, 6- or 7-indazolyl, 2-, 3-, 4-, 5-, 6-, 7- or 8-quinolyl, 1-, 3-, 4-, 5-, 6-, 7-, or 8-isoquinolyl, 2-, 4-, 5-, 6-, 7- or 8-quinazolinyl, 3-, 4-, 5-, 6-, 7- or 8-cinnolinyl, 2-, 3-, 5-, 6-, 7- or 8-quinoxalinyl, 1-, 4-, 5-, 6-, 7- or 8-phthalazinyl. The corresponding N-oxide these compounds, for example, 1-oxy-2-, 3- or 4-pyridyl, are also included.

Suitable heterocycles include thiophenyl and quinolyl.

Pyridyl may be 2-, 3- or 4-pyridyl. Thienyl may be 2- or 3-thienyl. Furyl may be 2- or 3-furyl.

Monosubstituted phenyl radicals may be substituted in the 2-, 3- or 4-position, disubstituted in the 2,3-, 2,4-, 2,5-, 2,6-, 3,4- or 3,5-position or trisubstituted in the 2,3,4-, 2,3,5-, 2,3,6-, 2,4,5-, 2,4,6- or 3,4,5-position. The same also applies in context to the N-containing heteroaromatics, the thiophene radicals or the furyl radicals.

In the case of di- or trisubstitution of a radical, the substituents may be identical or different.

The compounds of formula I include those containing one or more acidic or basic groups or one or more basic heterocycles, and the corresponding physiologically or toxicologically tolerated salts, in particular the pharmaceutically usable salts. Compounds of formula I which carry acidic groups, e.g., one or more COOH groups, may be provided as alkali metal salts, such as sodium or potassium salts; alkaline earth metal salts, such as calcium or magnesium salts; or ammonium salts, such as salts with ammonia or organic amines or amino acids. For compounds of formula I in which R3 is hydrogen, for example, deprotonation of the sulfonamide moiety to yield a sodium salt is possible. Compounds of formula I which contain one or more basic (i.e., protonatable) groups or contain one or more basic heterocyclic rings can also be used in the form of their physiologically tolerated acid addition salts with inorganic or organic acids, such as hydrochlorides, phosphates, sulfates, methanesulfonates, acetates, lactates, maleates, fumarates, malates, gluconates, etc. If the compounds of formula I contain both an acidic and a basic group, the compounds may be provided as inert salts, e.g., betaines, in addition to the other salt forms described above. Salts can be obtained from the compounds of formula I by conventional processes, for example, by combination with an acid or base in a solvent or dispersant or by anion exchange from other salts.

With appropriate substitution, the compounds of formula I may be present in stereoisomeric forms. If the compounds of formula I contain one or more centers of asymmetry, they may, independently of one another, have the S-configuration or the R-configuation. The invention relates to the use of all possible stereoisomers (e.g., enantiomers or diastereomers), and mixtures of two or more stereomeric forms (e.g. enantiomers and/or diastereomers), in any desired ratio. The invention thus relates, for example, to enantiomers in enantiomerically pure form, both as levorotatory and as dextrorotatory antipodes, and also in the form of mixtures of the two enantiomers in different ratios or in the form of racemates. The preparation of individual stereoisomers can be effected, if desired, by separation of a mixture by conventional methods or, for example, by stereoselective synthesis. In the presence of mobile hydrogen atoms, the present invention also comprises all tautomeric forms of the compounds of formula I.

The compounds of formula I can be prepared by different chemical processes, some of which are illustrated below in scheme 1 or 2. The radicals R(1) to R(7) used herein are defined above.

According to scheme 1, below, compounds according to the invention can be prepared by first reacting an aminocarboxylic acid of formula II, for example, with a sulfonyl chloride of formula R(4)—SO₂—Cl or a sulfonic acid anhydride, in a solvent such as water, pyridine or ether, in the presence of a base. Suitable bases include inorganic bases, such as sodium carbonate or potassium hydroxide; or organic bases, such as pyridine or triethylamine.

The resulting sulfonylaminocarboxylic acid of formula III can then be activated to give an acid chloride, for example, by reaction with a chlorinating agent such as phosphorus pentachloride, phosphorus oxychloride or thionyl chloride, in an inert solvent, followed by reaction with an amine of formula HNR(1)R(2) to give the title compounds of formula I. The activation of the carboxylic acid group in the compound of formula III can also be effected by numerous methods familiar to a person skilled in the art, which are used in peptide chemistry for forming amide bonds; for example, by conversion to a mixed anhydride or an activated ester, or with the use of a carbodiimide, such as dicyclohexylcarbodiimide.

The reaction of the activated sulfonylaminocarboxylic acid with an amine of formula HNR(1)R(2) is typically carried out in an inert solvent such as pyridine, tetrahydrofuran or toluene, with or without the addition of an inert auxiliary base (e.g., a tertiary amine or pyridine).

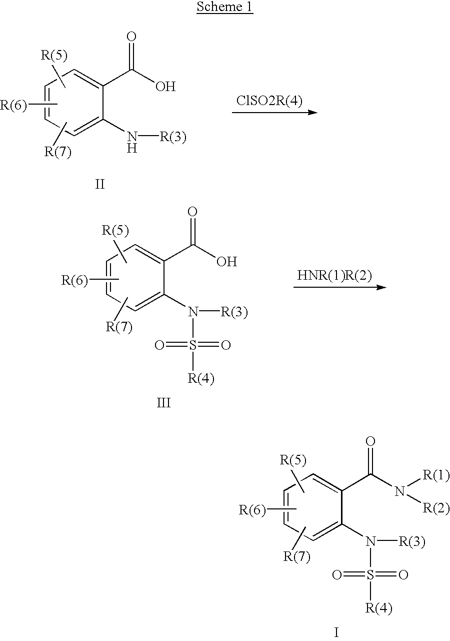

It is also possible, in accordance with scheme 2, below, to first react an anhydride of formula IV with an amine of formula HNR(1)R(2) to give an o-aminobenzamide of formula VII; this aminobenzamide is then reacted with a sulfonyl chloride of formula R(4)SO₂Cl to obtain a compound of formula I in which R(3) is hydrogen. Another possibility for preparing the intermediates of formula VII involves the amidation of an o-nitrobenzoic acid of formula V with an amine of formula HNR(1)R(2), followed by reduction of the nitro group to the amine.

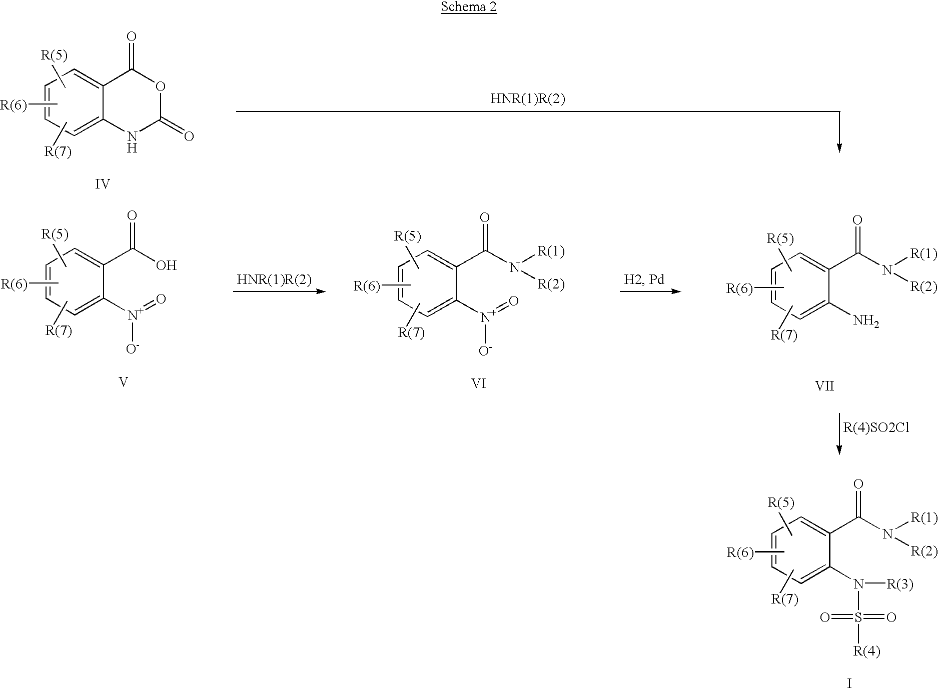

In any of these procedures, it may be appropriate to temporarily protect functional groups in the molecule during certain reaction steps. Such protective group techniques are familiar to a person skilled in the art. The choice of a suitable protective group, and the methods for their introduction and elimination, are described in the literature and can be adapted to the individual compounds without difficulties.

The compounds of formula I and their physiologically tolerated salts can be used as medicaments by themselves, in mixtures with one another, or in mixtures with other pharmaceutically active compounds. The compounds, or mixtures thereof, may be provided in the form of pharmaceutical formulations. Such formulations may be used as medicaments in animals such as mammals, and in particular to humans. The present invention also relates to the use of the compounds of formula I and their physiologically tolerated salts for the preparation of medicaments having a K⁺-channel-blocking action for the therapy and prophylaxis of the previously described conditions. According to the invention, pharmaceutical formulations may contain an active ingredient comprising an effective dose of at least one compound of formula I, and/or a physiologically tolerated salt thereof, in addition to conventional, pharmaceutically satisfactory carriers and excipients. The pharmaceutical formulations usually contain from 0.1 to 90 percent by weight of the compounds of formula I and/or their physiologically tolerated salts. The pharmaceutical formulations can be prepared in a manner known in the art. The compounds of formula I and/or their physiologically tolerated salts may be provided in a pharmaceutical composition comprising one or more solid or liquid pharmaceutical carriers and/or excipients, if desired. The pharmaceutical composition may further comprise other pharmaceutically active substances. The composition may be administered in a suitable dosage form in human or veterinary medicine.

Medicaments which contain compounds of formula I according to the invention and/or their physiologically tolerated salts can be administered orally, parenterally, intravenously, rectally, by inhalation or topically. The route of administration typically varies depending on the individual case, such as the particular profile of symptoms being treated.

Excipients which are suitable for the desired medicament formulation are familiar to a person skilled in the art. Solvents, gel formers, suppository bases, tabletting excipients and other active substance carriers, for example, antioxidants, dispersants, emulsifiers, antifoams, flavors, preservatives, solubilizers, agents for achieving a depot effect, buffer substances or colorants can be used.

The compounds of formula I can also be used in combination with other pharmaceutically active substances for achieving an advantageous therapeutic effect. For example, combinations with substances having cardiovascular activity are possible in the treatment of cardiovascular diseases. Suitable combination partners of this type include, for example, class I, class II or class III antiarrhythmic agents, such as IKₛ- or IKᵣ channel blockers (e.g., dofetilide); antihypertensive substances, such as ACE inhibitors (e.g., enalapril, captopril, ramipril); angiotensin antagonists; K⁺ channel activators; alpha- and beta-receptor blockers; sympathomimetic and adrenergic compounds; Na⁺/H⁺ exchange inhibitors; calcium channel antagonists; phosphodiesterase inhibitors; and other positively inotropic substances, such as digitalis glycosides or diuretics.

For an oral dosage form, the active compounds are mixed with the additives suitable for this purpose, such as carriers, stabilizers or inert diluents, and are administered in a suitable dosage form, such as tablets, coated tablets, capsules, or aqueous, alcoholic or oily solutions, by the conventional methods. Suitable inert carriers include, for example, gum arabic, magnesia, magnesium carbonate, potassium phosphate, lactose, glucose, or starch (e.g., corn starch). The formulation may be effected in the form of both dry granules and moist granules. Suitable oily carriers or suitable solvents include vegetable or animal oils, such as sunflower oil or cod liver oil. Suitable solvents for aqueous or alcoholic solutions include, for example, water, ethanol or sugar solutions, or mixtures thereof. Additional excipients include, for example, polyethylene glycols and polypropylene glycols.

For subcutaneous or intravenous administration, the active compounds may be provided in a solution suspension or emulsion with the substances customary for this purpose, such as solubilizers, emulsifiers or further excipients. The compounds of formula I, and their physiologically tolerated salts, can also be lyophilized. The lyophilisates obtained can be used, for example, in the preparation of injection or infusion dosage forms. Suitable solvents include, for example, water, physiological saline solution or alcohols (e.g., ethanol, propanol or glycerol), as well as sugar solutions, such as glucose or mannitol solutions, or mixtures of the various solvents mentioned.

Suitable dosage forms include aerosols and sprays. Such dosage forms include solutions, suspensions or emulsions of the active substances of formula I, or their physiologically tolerated salts, in a pharmaceutically acceptable solvent (e.g., ethanol or water) or a mixture of such solvents. The formulation can, if required, also contain other pharmaceutical excipients, such as surfactants, emulsifiers and stabilizers and a propellant. Such a formulation contains the active substance usually in a concentration of from about 0.1 to 10, optionally from about 0.3 to 3, percent by weight.

The dosage form of the active substance of formula I, or the physiologically tolerated salt thereof, should be adapted to the circumstances of the individual case in the customary manner for an optimum effect. Such circumstances include the frequency of administration, the potency and duration of action of the compounds used, the type and severity of the disease to be treated, the sex, age, weight and individual responsiveness of the person or animal being treated, and whether the dosage form is administered as a therapy or prophylactic. Usually, the daily dose of a compound of formula I for a patient weighing about 75 kg is from 0.001 mg/kg body weight to 100 mg/kg body weight, optionally from 0.01 mg/kg body weight to 20 mg/kg body weight. The dose can be administered in the form of a single dose or can be divided into a plurality of doses, e.g., two, three or four doses. In the treatment of acute cases of cardiac arrhythmias (e.g., in intensive care), parenteral administration by injection or infusion, for example, by continuous intravenous infusion, may be advantageous.

### EXAMPLES

### Example 1

Preparation of a Sulfonamide of Formula III (Scheme 1)

10 mmol of an anthranilic acid of formula II and 25 mmol of sodium carbonate were dissolved in 12 ml of water, and a total of 11 mmol of sulfonyl chloride ClSO₂R(4) were added in portions at 70° C. and stirred for 8 h. The mixture was cooled to 0° C. and the product of formula III was filtered off with suction, washed with cold water, and dried at 45° C. in a vacuum drying oven.

### Example 2

Preparation of the Acid Chlorides

8 mmol of the acid of formula III were suspended in 15 ml of dry toluene, and 9.6 mmol of phosphorus pentachloride were introduced slowly at room temperature. The mixture was stirred for 3 h at 50° C. and cooled to 0° C. The acid chloride was filtered off with suction, washed with a little toluene, and dried at 45° C. in a vacuum drying oven.

### Example 3

Preparation of the Anthranilamides of Formula I

3 mmol of the acid chloride were dissolved in 30 ml of dry dichloromethane, and 3 ml of triethylamine and 3.3 mmol of the amine NHR(1)R(2) were added. Stirring was effected for 18 h at room temperature, and the product was washed with water, dried over sodium sulfate and evaporated down. The anthranilamide of formula I was purified by chromatography, if required.

The compounds of table 1 (examples 1-32) were prepared in this manner.

### Example 4

Pharmacological Activity of the Prepared Compounds

Human Kv1.5 channels were expressed in xenopus oocytes. For this purpose, oocytes from Xenopus laevis were first isolated and defolliculated. Kv1.5 coding RNA synthesized in vitro was then injected into these oocytes. After Kv1.5 protein expression for 1-7 days, Kv1.5 currents were measured in the oocytes using the two-microelectrode voltage clamp technique. The Kv1.5 channels were activated here with 500 ms voltage jumps to 0 mV and 40 mV. The bath was flushed with a solution of the following: NaCl 96 mM, KCl 2 mM, CaCl₂ 1.8 mM, MgCl₂ 1 mM, HEPES 5 mM (titrated with NaOH to pH 7.4). These experiments were carried out at room temperature. The following were used for data collection and analysis: Geneclamp amplifier (Axon Instruments, Foster City, USA) and MacLab D/A converter and software (ADInstruments, Castle Hill, Australia). The compounds according to the invention were tested by adding them to the bath solution in different concentrations. The effects of the compounds were calculated as percentage inhibition of the Kv1.5 control current which was obtained when no compound was added to the solution. The data were then extrapolated using the Hill equation, in order to determine the inhibitory concentrations (IC₅₀) for the respective compounds. The IC₅₀ values obtained are shown in table 1.

TABLE 1
IC-₅₀ values for exemplary compounds

| Example | | | |
|---|---|---|---|
| No. | Structure | MS (ES+): m/z = | IC₅₀ [μM] |
| 1 | 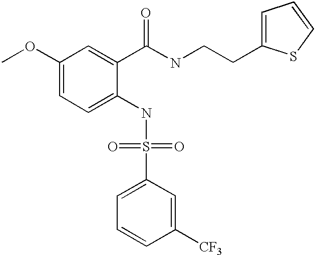 | 485 | 3.0 |
| 2 | 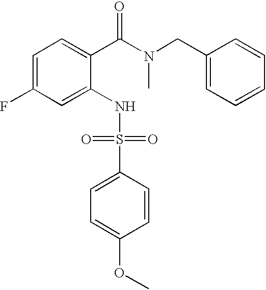 | 429 | 4.1 |
| 3 | 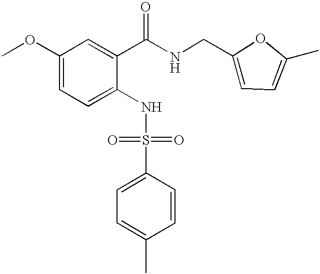 | 415 | 2.1 |
| 4 | 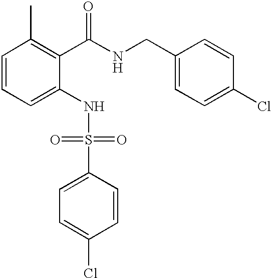 | 449 | 2.4 |
| 5 | 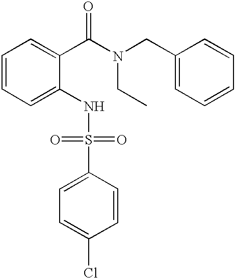 | 429 | 3.2 |
| 6 | 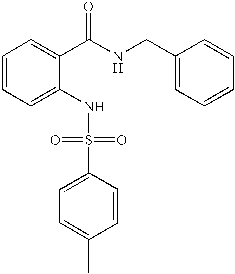 | 381 | 4.1 |
| 7 | 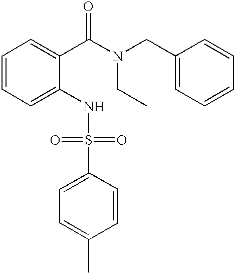 | 409 | 5.0 |
| 8 | 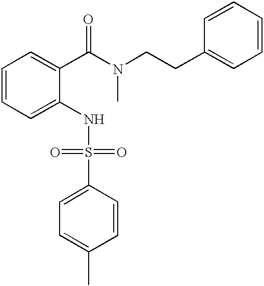 | 409 | 4.6 |
| 9 | 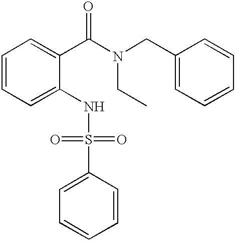 | 395 | 4.7 |
| 10 | 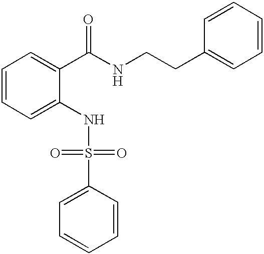 | 381 | 3.8 |
| 11 | 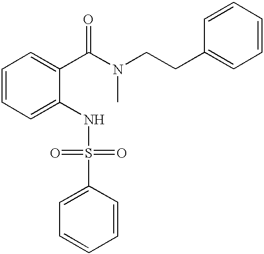 | 395 | 3.7 |
| 12 | 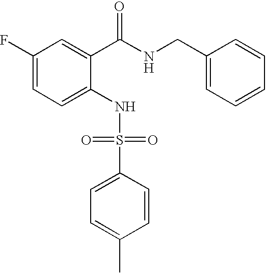 | 399 | 3.6 |
| 13 | 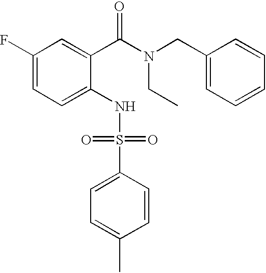 | 427 | 3.7 |
| 14 | 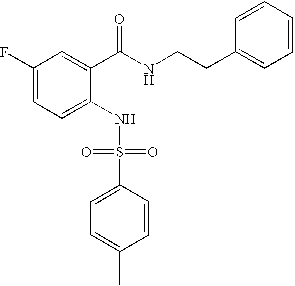 | 413 | 4.1 |
| 15 | 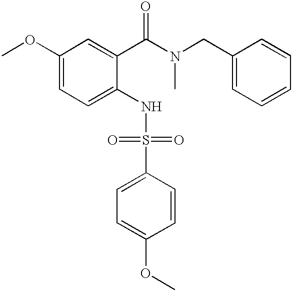 | 441 | 4.5 |
| 16 | 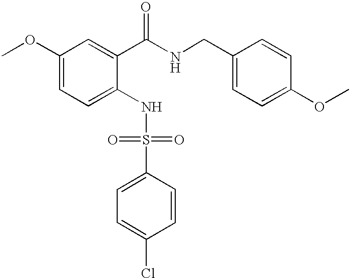 | 461 | 3.3 |
| 17 | 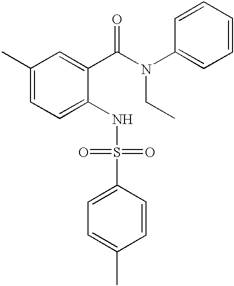 | 409 | 1.6 |
| 18 | 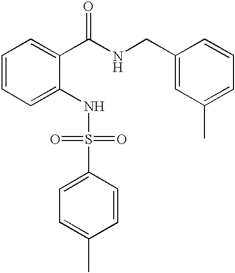 | 395 | 3.5 |
| 19 | 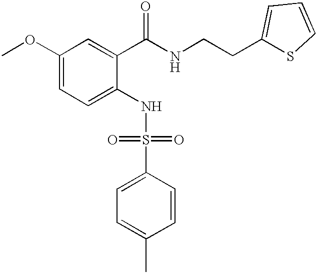 | 431 | 2.6 |
| 20 | 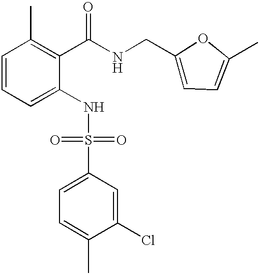 | 433 | 3.8 |
| 21 | 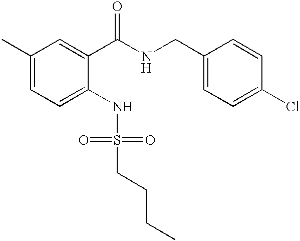 | 395 | 6.1 |
| 22 | 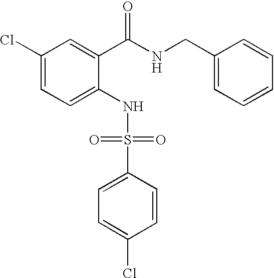 | 435 | 6.0 |
| 23 | 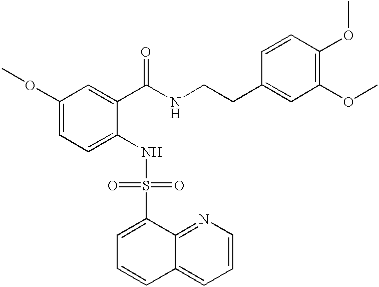 | 522 | 6.9 |
| 24 | 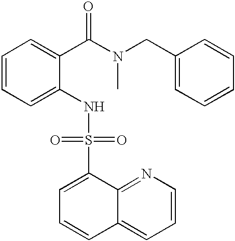 | 432 | 4.6 |
| 25 | 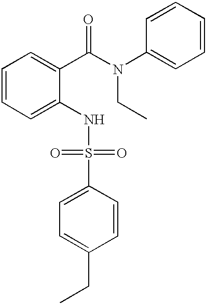 | 409 | 4.2 |
| 26 | 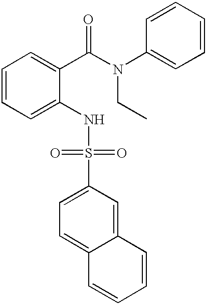 | 431 | 5.6 |
| 27 | 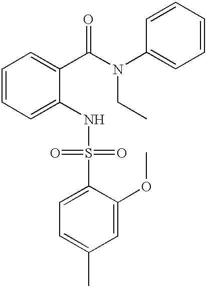 | 425 | 3.0 |
| 28 | 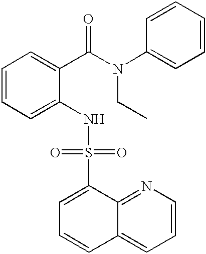 | 432 | 1.2 |
| 29 | 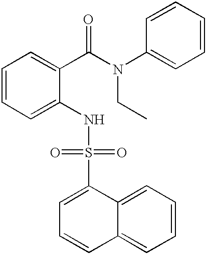 | 431 | 1.9 |
| 30 | 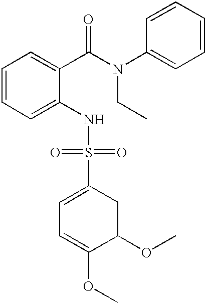 | 441 | 3.5 |
| 31 | 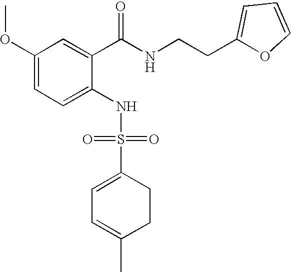 | 415 | 3.2 |
| 32 | 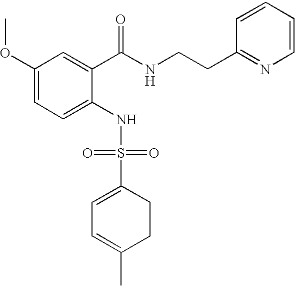 | 426 | 8.0 |

We claim

1. A method of treating a medical condition characterized by abnormal K⁺-channel-activity comprising administering one or more compounds of formula I 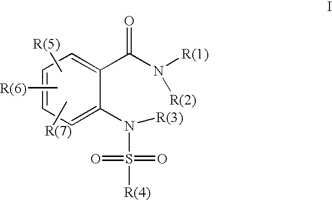
in which:
R(1) is (CH₂)ₓ—R(8)
x is 0, 1, 2, 3, 4 or 5;
R(8) is phenyl, thienyl or furanyl, phenyl, thienyl and furanyl being unsubstituted or substituted by 1, 2 or 3 substituents chosen from among F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(2) is hydrogen or alkyl having 1, 2 or 3 carbon atoms;
R(3) is hydrogen or alkyl having 1, 2 or 3 carbon atoms;
R(4) is alkyl having 3, 4, 5, 6 or 7 carbon atoms, cycloalkyl having 3, 4, 5, 6 or 7 carbon atoms, phenyl, naphthyl or heteroaryl, phenyl, naphthyl and heteroaryl being unsubstituted or substituted by 1, 2 or 3 substituents chosen from among F, Cl, Br, I, CF₃, OCF₃, NO₂, COOMe, CONH₂, COMe, NH₂, OH, alkyl having 1, 2, 3 or 4 carbon atoms, alkoxy having 1, 2, 3 or 4 carbon atoms, dimethylamino, methylsulfonyl and methylsulfonylamino;
R(5), R(6) and R(7), independently of one another, are chosen from among F, Cl, Br, I, CF₃, OCF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
or any pharmaceutically acceptable salt thereof, to a patient in need of such a treatment.

2. The method of claim 1, wherein:
R(1) is (CH₂)ₓ—R(8)
x is 1, 2, 3 or 4;
R(8) is phenyl, thienyl or furanyl, phenyl, thienyl and furanyl being unsubstituted or substituted by 1, 2 or 3 substituents chosen from among F, Cl, Br, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(2) is hydrogen or alkyl having 1, 2 or 3 carbon atoms;
R(3) is hydrogen or alkyl having 1, 2 or 3 carbon atoms;
R(4) is alkyl having 4, 5, 6 or 7 carbon atoms, cycloalkyl having 5, 6 or 7 carbon atoms, phenyl or heteroaryl, phenyl and heteroaryl being unsubstituted or substituted by 1, 2 or 3 substituents chosen from among F, Cl, Br, CF₃, OCF₃, NO₂, COOMe, CONH₂, COMe, NH₂, OH, alkyl having 1, 2, 3 or 4 carbon atoms, alkoxy having 1, 2, 3 or 4 carbon atoms, dimethylamino, methylsulfonyl and methylsulfonylamino;
R(5), R(6) and R(7), independently of one another, are chosen from among F, Cl, Br, CF₃, OCF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino; or any pharmaceutically acceptable salts thereof.

3. The method of claim 1, wherein:
R(1) is (CH₂)ₓ—R(8)
x is 1, 2, 3 or 4;
R(8) is phenyl, thienyl or furanyl, phenyl, thienyl and furanyl being unsubstituted or substituted by 1, 2 or 3 substituents chosen from among F, Cl, Br, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(2) is hydrogen or alkyl having 1, 2 or 3 carbon atoms;
R(3) is hydrogen or alkyl having 1 or 2 carbon atoms;
R(4) is phenyl, phenyl being unsubstituted or substituted by 1, 2 or 3 substituents chosen from among F, Cl, Br, CF₃, OCF₃, NO₂, COOMe, CONH₂, COMe, NH₂, OH, alkyl having 1, 2, 3 or 4 carbon atoms, alkoxy having 1, 2, 3 or 4 carbon atoms, dimethylamino, methylsulfonyl and methylsulfonylamino;
R(5), R(6) and R(7), independently of one another, are chosen from among F, Cl, Br, CF₃, OCF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
and any pharmaceutically acceptable salts thereof.

4. The method of claim 1, wherein:
R(1) is (CH₂)ₓ—R(8)
x is 1 or 2;
R(8) is phenyl, thienyl or furanyl, phenyl, thienyl and furanyl being unsubstituted or substituted by 1, 2 or 3 substituents chosen from among F, Cl, Br, CF₃, OCF₃, CN, COOMe, CONH₂, COMe, alkyl having 1, 2 or 3 carbon atoms, alkoxy having 1, 2 or 3 carbon atoms, sulfamoyl, methylsulfonyl and methylsulfonylamino;
R(2) is hydrogen or alkyl having 1 or 2 carbon atoms;
R(3) is hydrogen;
R(4) is phenyl, phenyl being unsubstituted or substituted by 1, 2 or 3 substituents chosen from among F, Cl, Br, CF₃, OCF₃, COMe, alkyl having 1, 2 or 3 carbon atoms and alkoxy having 1, 2 or 3 carbon atoms;
R(5), R(6) and R(7), independently of one another, are chosen from among F, Cl, Br, CF₃, OCF₃, CN, COOMe, CONH₂, COMe, alkyl having 1, 2 or 3 carbon atoms, alkoxy having 1, 2, 3 or 4 carbon atoms, dimethylamino, sulfamoyl, methylsulfonyl and methylsulfonylamino;
and any pharmaceutically acceptable salts thereof.

5. The method of claim 1, wherein one or more compounds of formula I are provided in a pharmaceutical composition.

6. The method of claim 1, wherein the medical condition is a cardiac arrhythmia.

7. The method of claim 1, wherein the medical condition is a reentry arrhythmia.

8. The method of claim 1, wherein the medical condition is a supraventricular arrhythmia.

9. The method of claim 1, wherein the medical condition is an atrial fibrillation or atrial flutter.

10. The method of claim 1, wherein the compound of formula I is used to terminate an atrial fibrillation or atrial flutter (cardioversion).

11. The method of claim 5, wherein the pharmaceutical composition comprises an active amount of at least one compound of formula I, or a pharmaceutically acceptable salt thereof, and one or more pharmaceutically acceptable carriers or additives.

12. The method of claim 5, wherein the pharmaceutical composition comprises an active amount of at least one compound of formula I, or a pharmaceutically acceptable salt thereof, and one or more additional pharmaceutically active compounds.

13. A method of preventing a medical condition characterized by abnormal K⁺-channel-activity comprising administering one or more compounds of formula I 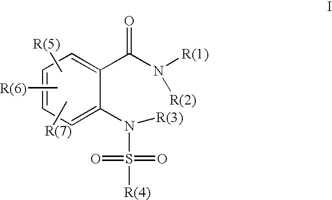
in which:
R(1) is (CH₂)ₓ—R(8)
x is 0, 1, 2, 3, 4 or 5;
R(8) is phenyl, thienyl or furanyl, phenyl, thienyl and furanyl being unsubstituted or substituted by 1, 2 or 3 substituents chosen from among F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(2) is hydrogen or alkyl having 1, 2 or 3 carbon atoms;
R(3) is hydrogen or alkyl having 1, 2 or 3 carbon atoms;
R(4) is alkyl having 3, 4, 5, 6 or 7 carbon atoms, cycloalkyl having 3, 4, 5, 6 or 7 carbon atoms, phenyl, naphthyl or heteroaryl, phenyl, naphthyl and heteroaryl being unsubstituted or substituted by 1, 2 or 3 substituents chosen from among F, Cl, Br, I, CF₃, OCF₃, NO₂, COOMe, CONH₂, COMe, NH₂, OH, alkyl having 1, 2, 3 or 4 carbon atoms, alkoxy having 1, 2, 3 or 4 carbon atoms, dimethylamino, methylsulfonyl and methylsulfonylamino;
R(5), R(6) and R(7), independently of one another, are chosen from among F, Cl, Br, I, CF₃, OCF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
or any pharmaceutically acceptable salt thereof, to a patient in need of such a treatment.

14. The method of claim 13, wherein:
R(1) is (CH₂)ₓ—R(8)
x is 1, 2, 3 or 4;
R(8) is phenyl, thienyl or furanyl, phenyl, thienyl and furanyl being unsubstituted or substituted by 1, 2 or 3 substituents chosen from among F, Cl, Br, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(2) is hydrogen or alkyl having 1, 2 or 3 carbon atoms;
R(3) is hydrogen or alkyl having 1, 2 or 3 carbon atoms;
R(4) is alkyl having 4, 5, 6 or 7 carbon atoms, cycloalkyl having 5, 6 or 7 carbon atoms, phenyl or heteroaryl, phenyl and heteroaryl being unsubstituted or substituted by 1, 2 or 3 substituents chosen from among F, Cl, Br, CF₃, OCF₃, NO₂, COOMe, CONH₂, COMe, NH₂, OH, alkyl having 1, 2, 3 or 4 carbon atoms, alkoxy having 1, 2, 3 or 4 carbon atoms, dimethylamino, methylsulfonyl and methylsulfonylamino;
R(5), R(6) and R(7), independently of one another, are chosen from among F, Cl, Br, CF₃, OCF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
or any pharmaceutically acceptable salts thereof.

15. The method of claim 13, wherein:
R(1) is (CH₂)ₓ—R(8)
x is 1, 2, 3 or 4;
R(8) is phenyl, thienyl or furanyl, phenyl, thienyl and furanyl being unsubstituted or substituted by 1, 2 or 3 substituents chosen from among F, Cl, Br, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(2) is hydrogen or alkyl having 1, 2 or 3 carbon atoms;
R(3) is hydrogen or alkyl having 1 or 2 carbon atoms;
R(4) is phenyl, phenyl being unsubstituted or substituted by 1, 2 or 3 substituents chosen from among F, Cl, Br, CF₃, OCF₃, NO₂, COOMe, CONH₂, COMe, NH₂, OH, alkyl having 1, 2, 3 or 4 carbon atoms, alkoxy having 1, 2, 3 or 4 carbon atoms, dimethylamino, methylsulfonyl and methylsulfonylamino;
R(5), R(6) and R(7), independently of one another, are chosen from among F, Cl, Br, CF₃, OCF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
and any pharmaceutically acceptable salts thereof.

16. The method of claim 13, wherein:
R(1) is (CH₂)ₓ—R(8)
x is 1 or 2;
R(8) is phenyl, thienyl or furanyl, phenyl, thienyl and furanyl being unsubstituted or substituted by 1, 2 or 3 substituents chosen from among F, Cl, Br, CF₃, OCF₃, CN, COOMe, CONH₂, COMe, alkyl having 1, 2 or 3 carbon atoms, alkoxy having 1, 2 or 3 carbon atoms, sulfamoyl, methylsulfonyl and methylsulfonylamino;
R(2) is hydrogen or alkyl having 1 or 2 carbon atoms;
R(3) is hydrogen;
R(4) is phenyl, phenyl being unsubstituted or substituted by 1, 2 or 3 substituents chosen from among F, Cl, Br, CF₃, OCF₃, COMe, alkyl having 1, 2 or 3 carbon atoms and alkoxy having 1, 2 or 3 carbon atoms;
R(5), R(6) and R(7), independently of one another, are chosen from among F, Cl, Br, CF₃, OCF₃, CN, COOMe, CONH₂, COMe, alkyl having 1, 2 or 3 carbon atoms, alkoxy having 1, 2, 3 or 4 carbon atoms, dimethylamino, sulfamoyl, methylsulfonyl and methylsulfonylamino;
and any pharmaceutically acceptable salts thereof.

17. The method of claim 13, wherein one or more compounds of formula I are provided in a pharmaceutical composition.

18. The method of claim 13, wherein the medical condition is a cardiac arrhythmia.

19. The method of claim 13, wherein the medical condition is a reentry arrhythmia.

20. The method of claim 13, wherein the medical condition is a supraventricular arrhythmia.

21. The method of claim 13, wherein the medical condition is an atrial fibrillation or atrial flutter.

22. The method of claim 17, wherein the pharmaceutical composition comprises an active amount of at least one compound of formula I, or a pharmaceutically acceptable salt thereof, and one or more pharmaceutically acceptable carriers or additives.

23. The method of claim 17, wherein the pharmaceutical composition comprises an active amount of at least one compound of formula I, or a pharmaceutically acceptable salt thereof, and one or more additional pharmaceutically active compounds.
